(19) United States
(12) Patent Application Publication
(10) Pub. No.: US 2021/0307651 A1
(43) Pub. Date: Oct. 7, 2021

(54) OBJECTIVE RANGE OF MOTION MONITORING

(71) Applicant: CIPHER SKIN, Denver, CO (US)

(72) Inventors: Phillip Bogdanovich, Denver, CO (US); Guido Gioberto, Denver, CO (US); Shaka Bahadu, Emeryville, CA (US); Craig Weller, Evergreen, CO (US)

(73) Assignee: CIPHER SKIN, Denver, CO (US)

(21) Appl. No.: 17/217,503

(22) Filed: Mar. 30, 2021

Related U.S. Application Data
(60) Provisional application No. 63/003,705, filed on Apr. 1, 2020.

(51) Int. Cl. A61B 5/11; A61B 5/00

(52) U.S. Cl. CPC A61B 5/1121; A61B 5/0024; A61B 5/742; A61B 5/6801

(57) ABSTRACT

A motion monitoring system includes a wearable monitoring device configured to be worn on a human body that includes a body part. The wearable monitoring device is deformable with movement of the body part. The wearable monitoring device includes a sensor system configured to deform with movement of the body part. The sensor system is configured to detect deformation occurring with movement of the body part. Furthermore, the motion monitoring system includes a user device that is in communication with the wearable monitoring device. The user device is capable of receiving data from the sensor system that corresponds to the detected deformation occurring with movement of the body part.

### CROSS-REFERENCE TO RELATED APPLICATION

This application claims the benefit of U.S. Provisional Application No. 63/003,705 filed Apr. 1, 2020.

### FIELD OF THE INVENTION

The present application relates to systems and methods for objectively measuring range of motion, and more particularly measuring range of motion of a human body part. The present application may be useful for anyone attempting to measure the range of motion of a body part, including but not limited to physical therapists, doctors, nurses, athletic trainers, and individuals.

### BACKGROUND

Diagnosis and treatment of injuries to body parts often involves measurement of the range of motion of a body part. Often times, the range of motion of an injured body part and a similar body part (i.e. left and right arms) is compared to determine the severity of the injury and the progress made during treatment for an injury. Additionally, trainers and medical professionals may determine the range of motion of a body part to design exercises to help prevent injury to a limb or other body part. For example only, a limited range of mobility in a shoulder joint may cause injury to the elbow on the same arm.

Current measurement of range of motion of a body part requires a practitioner to either estimate the range of motion based on experience and practice, or use large protractors to measure certain types of range of motion. Both methods include significant amounts of error and are subject to inconsistencies between practitioners. Both methods also require the practitioner to record his or her results on paper rather than digitally. Additionally, neither method allows for a user to perform range of motion determinations on their own or to view a visual representation of the range of motion. Thus, a digital and accurate apparatus and method for measuring range of motion that could be used by either a practitioner or individual patient is needed.

### SUMMARY

This present disclosure relates to range of motion measurement devices and techniques, in particular measurement of range of motion of the human body, including but not limited to limbs such as arms and legs, the spine, pelvis, and any other body part. The device may include a sleeve or sleeves that are placed on the body part and wirelessly communicate with a user device. The user device includes a program or application that may instruct the wearer what motions to perform with his or her body parts, records and stores the measurements of those movements, and creates a visual depiction of the movement of the body part. The program or application may also allow a user to move on his or her own and then record and store the measurements of those movements and create a visual depiction of the movement. The user device may wirelessly communicate with a display device that displays the visual depiction of the movement of the body part. The data received by the user device may be stored in the user device, in the cloud, and/or in any data storage device or central database.

A system may include a sleeve or sleeves configured to be placed on a human body that includes a sensor configured to contact the skin and a user device in wireless communication with the sleeve that is capable of receiving and storing data communicated from the sleeve. The user device may be capable of creating a visual rendering of the movement of a body part using data communicated from the sleeve. The system may also include a display device, wherein the display device is in wireless communication with the user device. The display device may also display the visual rendering. The user device may also be in wireless communication with a storage device, the storage device storing data wirelessly transmitted from the user device. The system may also include a battery compartment disposed on the sleeve and a battery component configured to fit within the battery compartment. The system may also include a battery life indicator disposed on the battery.

A method for determining the range of motion of a human body may include receiving data from a sleeve placed on the human body, said data including angular rotation and direction, calculating the range of motion data, storing the range of motion data, and generating a visual depiction of the movement from the data. The method may also include storing the range of motion in the cloud or a central database. The method may also include directing the user to move the human body part in a particular way. The method may also include storing data from previous range of motion determinations. The method may also include comparing previous range of motion determinations with a current range of motion determination.

In one embodiment, a motion monitoring system is disclosed that includes a wearable monitoring device configured to be worn on a human body that includes a body part. The wearable monitoring device is deformable with movement of the body part. The wearable monitoring device includes a sensor system configured to deform with movement of the body part. The sensor system is configured to detect deformation occurring with movement of the body part. Furthermore, the motion monitoring system includes a user device that is in communication with the wearable monitoring device. The user device is capable of receiving data from the sensor system that corresponds to the detected deformation occurring with movement of the body part.

In another embodiment, a method for determining the range of motion of a human body part is disclosed. The method includes detecting, by a sensor system of a monitoring device that is worn on a human body that includes a body part, deformation of the sensor system occurring with movement of the body part. The method also includes receiving data by a user device from a wearable monitoring device. The data corresponds to the detected deformation occurring with movement of the body part. Furthermore, the method includes visually rendering, with a display device, movement of the body part according to the received data.

### BRIEF DESCRIPTION OF THE DRAWINGS

FIG. 1 is a depiction of a range of motion monitoring system.

FIG. 2 is a plan view of a monitoring sleeve.

FIG. 3 is a flow chart describing a method of measuring range of motion using a range of motion monitoring system.

FIG. 4 is a depiction of another embodiment of a monitoring sleeve formed in the shape of a shirt.

FIG. 5 is a depiction of another embodiment of a monitoring sleeve formed in the shape of a pair of pants.

FIG. 6 is a depiction of another embodiment of a monitoring sleeve formed in the shape of a bodysuit.

FIG. 7 is a plan view of a lattice of a monitoring system of the monitoring sleeve of FIGS. 1-2 and 4-6.

FIG. 8 is a plan view of the lattice of FIG. 7 included on the monitoring sleeve of FIGS. 1-2 and 4-6.

### DETAILED DESCRIPTION

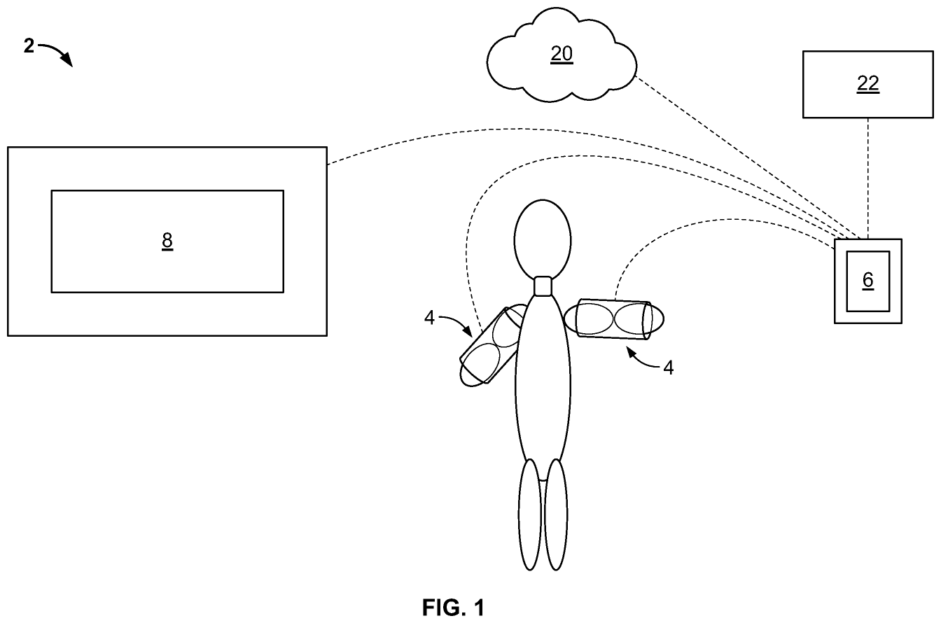

As shown in FIG. 1, one embodiment of an Objective Range of Motion Monitoring System 2 may be made up of one or more wearable monitoring devices, such as monitoring sleeves 4 that can be worn on the arm, shoulder, hip, knee, neck, or other joint of the human body. The monitoring system 2 may also include a user device 6 and a display device 8. The user device 6 may be wirelessly connected to a monitoring sleeve 4 such that data may be transferred to the user device 6. Additionally, the user device 6 and display device 8 may also be wirelessly connected such that images may be transmitted from the user device 6 to the display device 8.

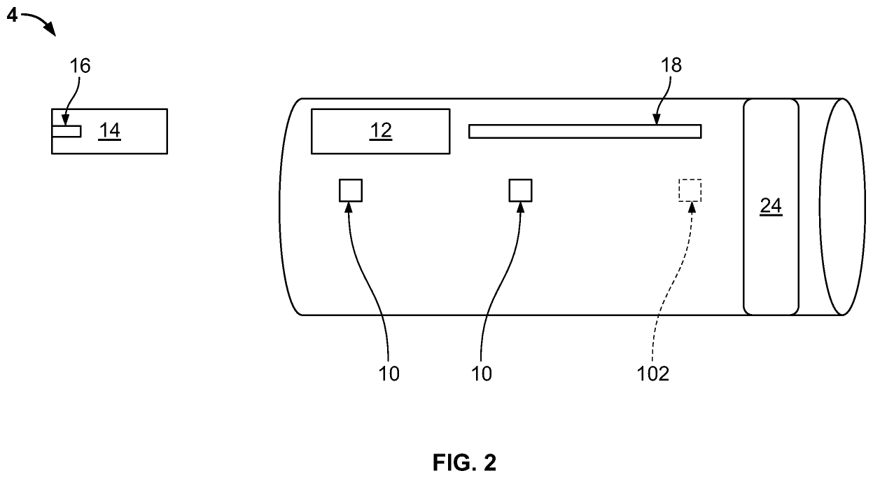

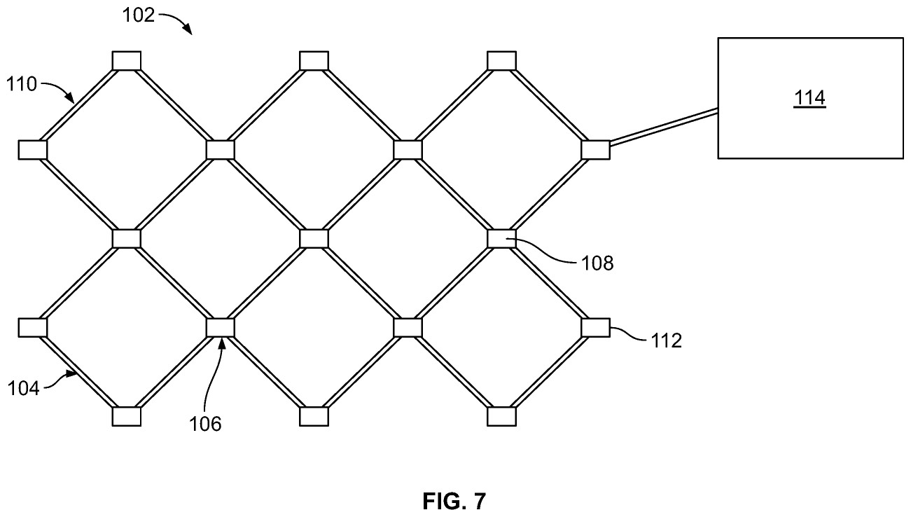

As shown in FIG. 2, a monitoring sleeve 4 may be made up of a stretchable fabric and may include at least one orientation sensor 10 integrated into the fabric, a battery compartment 12, a battery component 14, a battery life indicator 16, and an orientation indicator 18. A monitoring sleeve 4 may also include other sensors monitoring any bio-signals, including but not limited to oxygen levels, heart rate, respiration, etc. In another embodiment of an Objective Range of Motion Monitoring System, the monitoring sleeve 4 may include a geometric sensor system 102, which is illustrated in detail in FIG. 7 according to example embodiments of the present disclosure.

As shown in FIG. 7, the geometric sensor system 102 may be made up of at least one lattice 104, lattice intersections 106, a primary sensor 110, secondary sensors 112, and a monitoring system 114. The lattice 104 may be made of a flexible and deformable material that transmits both power and data and may be constructed of an array of repeating geometric patterns (e.g., an array of diamond-shaped, rectangular, parallelogram-shaped, or other polygonal patterns). The size of the lattice 104 may be determined by the limitations resulting from the relationship between the surface area to be monitored by the lattice 104 and the conductive, electrical, and physical properties of the lattice 104. Intersections 106 may be disposed at the junctions of the lattice 104.

The lattice 104 may be constructed and formed using the primary sensor 110 and at least one secondary sensor 112. A primary sensor 110 forms the layout and design on the lattice 104 and may be configured to integrate with one or more secondary sensors 112.

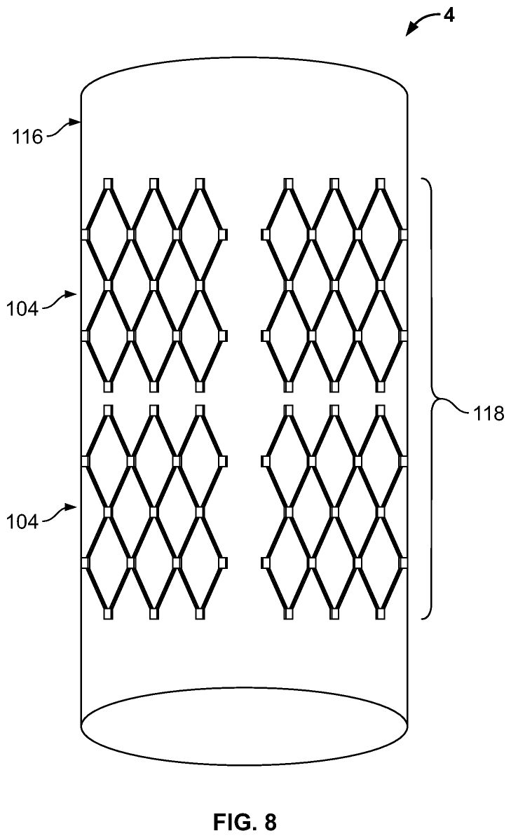

For example, the one or more secondary sensors 112 may be disposed at any intersection 106 of the primary sensor 110 of the lattice 104. The entire lattice 104 may be electronically connected to a monitoring system 114 which may accept and process both power and data transmissions from the lattice 104. A series of lattices 104 can be connected to cover great distances or span massive surfaces. A series of connected latices 104 comprises a super lattice 118, which may be included on the sleeve 4 as shown in FIG. 8.

By way of example, FIG. 8 shows four lattices 104 in operation. The lattices 104 may be deployed on a base object 116 (e.g., the stretchable fabric) of the sleeve 4 to collectively form a super lattice 118.

The sensor system 102 within the sleeve 4 may be configured to monitor the status of the wearer's body. The sensor system 102 may monitor for a variety of conditions, including strain, temperature, motion, position in space, flow, oxygen saturation, vascular lag, wall thickness, etc. It will be understood that the primary sensors 110 and secondary sensors 112 may monitor for any condition capable of being monitored with sensors.

The primary sensor 110 may most often be the mode of connection to the base object 116 being monitored. The primary sensor 110 may be capable of measuring for a specific series of changes in the base object 116 that is being monitored. For example, the primary sensor 110 may be used to monitor changes in electrical resistance to determine Freedom and Constraint Topology (“FACT”) on the surface of a base object 116, allowing the determination of changes in deflection, shearing force, and general motion with respect to the base object 116. As an example, the primary sensor 110 measures changes in voltage across the primary sensor 110 to determine a change in the length of base object 116 (i.e., an amount of deformation of the base object 116) and/or motion of the base object 116. A 3D model may be built based on these measured changes in resistance, for example, to track the movements of the wearer. In certain embodiments, the primary sensor 110 may also be capable of powering and transmitting data for discrete secondary sensors 112 and may also be scalable. The size and shape of the primary sensor 110 may be utilized to determine the size and shape of the lattice 104. Because the primary sensor 110 may be comprised of an array of repeating geometric patterns, the lattice 104 and super lattice 118 structures may be constructed very quickly with known electrical and conductive properties.

The secondary sensors 112 may be integrated into the primary sensor 110 to create a sensor-within-a-sensor platform. The secondary sensors 112 may measure specific metrics, including, for example, motion, temperature, or flow rate. In some embodiments, the secondary sensors 112 may be included at one or more conductive tabs 108 (FIG. 7). Conductive tabs 108 may be comprised of any conductive material. The conductive tabs 108 may measure any condition, including but not limited to strain, heat, motion, position in space, etc. The conductive tabs 108 may be preferably made of gold, however any other suitable material may be utilized. In certain embodiments, the secondary sensors 112 may not accept power and data over the same transmission lines of the lattice 104. In some embodiments, the secondary sensors 112 may be configured to monitor for conditions that cannot be monitored by the primary sensor 110.

The sensor system 102 may be modular in that each component part of the system 102 may be independently removed, changed, replaced, or repaired. For example, a section of the primary sensor 110 may be removed from the main body of the primary sensor 110, secondary sensors 112 may be removed or replaced, sections of lattice 104 may be removed or replaced, and sections within a super lattice 118 may be removed or replaced. The sensor system 102 may be configured to maintain functionality when components are removed regardless of the size of the system. Additionally, removing sensor components within the sensor system 102 does not impede the flow of electricity or data. Also, in certain embodiments, the sensor system 102 may allow for new components to be integrated because no portion of any layer within the system is a closed loop.

In use, the sensor system 102 allows for the monitoring of 3-dimensional objects (e.g., the sleeve 4) that may be complex in shape and/or massive in size or length. Using the sensor system 102, the sleeve 4 may be tessellated, or covered in repeated geometric patterns of identical interlocking shapes, which allows the surface of the sleeve 4 to be approximated in two dimensions. The approximated surface area of the 3-D object determines the geometric shape of the lattice 104 and primary sensor 110 and the size and number of segments in a single lattice 104/primary sensor 110. Frequency of secondary sensor 112 placement may be also determined by the specific monitoring needs related to the base object 116.

In certain embodiments, the sensor system 102 may monitor the metrics collected by both the primary sensors 110 and secondary sensors 112 on the same system clock at monitoring system 114. All of the data from the primary sensor 110 and secondary sensors 112 may be transmitted to the monitoring system 114 at the same time. This allows for less data processing at the monitoring system 114, which in turn provides faster data access and faster feedback regarding the monitoring of base object 116. Signals may also be integrated in a straightforward manner. Sensor output may be collated, analyzed as a time series by simple linear models, and used to build predictive models of events relevant to the monitored environment (injury, structural or catastrophic failure, etc.) Because the sensor system 102 is modular, it allows for the introduction of new secondary sensors 112 into the system as needed without redesigning the entire system or adding a new circuit to the system. If the size or portion of the base object 116 to be monitored changes, lattices 104 may be added or removed from the system as necessary.

Using the sensor system 102, the 3-D surface of the sleeve 4 may be transformed into planes which provide an active monitoring area. For example, if the sleeve 4 is worn on the user's arm, the arm may be approximated as the curved surface of a cylinder using the formula A=2πrh. The surface area determines the geometric shape of the primary sensor 110 and the distance between secondary sensors 112. The relationship between shape and surface area determines the number of segments in a single cell, hence the number of repeating geometric cells over a given area.

Accordingly, if one or more sleeves 4 are worn on body parts as represented in FIG. 1, the sensor system 102 can be used to detect how the body parts interact with the world perpetually and in real time. The sensor system 102 may be used to monitor multiple metrics simultaneously, on the same system clock.

The sensor system 102 may generate various signals, such as (in increasing order of complexity): 1) accurate monitoring of the desired shape as a time series; 2) complex instantaneous measurements by combining signals (for instance, measuring electrical to vascular lag as a proxy for vascular stiffness, or monitoring wall thickness and ambient temperature, the integration of (1) and (2) into near-instantaneous monitoring or user feedback, and the eventual acquisition of a sufficient database of integrated signals to form models for predicting certain events.

By way of example, the lattice 104 may have a parabolic shape and may be incorporated into the cylindrical sleeve 4. The sensor system 102 may monitor multiple types of strain across multiple planes, as well as temperature, flow, and expansion. These measurements may be made on the same clock and the output may be sent to a common table. Accordingly, the data should be gathered consistently (i.e., without “holes”).

As shown in FIG. 1, the monitoring sleeve 4 may be configured to fit over the limb or other body portion of an animal, including an arm, leg, or torso of a human being. The monitoring sleeve 4 may be positioned over and/or wrap around a joint (e.g., an elbow, shoulder, neck, ankle, hip, knee, hand, or foot joint, etc.). As the user flexes the joint (e.g., extends the joint, changes the angle of the joint) or otherwise initiates bodily movement, the sleeve 4 may deform, and the sensor system 102 may, in turn, detect and track flexure of that joint. In additional embodiments, the sleeve 4 may be positioned proximate a joint (e.g., a shoulder) without necessarily wrapping around that joint. As that joint is flexed, the sleeve 4 may move in space, and the sensor system 102 may, in turn, may detect and track this movement.

Furthermore, the orientation indicator 18 (FIG. 2) may allow a user or practitioner to properly orient the monitoring sleeve 4 by using markings and/or physical cups, sleeves, or any other indicator that a monitoring sleeve 4 is oriented properly. The battery compartment 12 may be located on the lateral side of the monitoring sleeve 4 and may be configured to accept a battery component 14. The battery 14 may include a battery life indicator 16 that indicates when the battery is installed in the battery compartment 12 such that the battery 14 may provide power to a monitoring sleeve 4 and when the battery 14 is low on power. A monitoring sleeve 4 may include flaps 24 that secure the sleeve to the body part and ensure contact between the skin of the wearer and the sensors.

Referring again to FIG. 1, the display device 8 may be any display capable of wirelessly connecting to the user device 6 and displaying a real-time image generated by the user device 6. The display device 8 may be any television, monitor, or other device that may connect wirelessly to the user device 6 via wireless internet, Bluetooth, or any other means of wirelessly connecting the display and user devices.

The user device 6 may be any device capable of running a program or application, receiving inputs from a practitioner, and wirelessly connecting to and receiving inputs from the monitoring sleeve 4. For example, and without limitation, a user device 6 could be a tablet, laptop, or smart phone.

The user device 6 may include a program or application that receives data from the sensor system 102 of the monitoring sleeve 4, calculates a range of motion from the data, and creates a real-time visual model of range of motion of a body part as the wearer of the sleeve 4 moves the body part. The user device 6 may also receive inputs from a practitioner or user regarding the wearer's personal information, information regarding which body part is being tested, and any diagnostic information regarding the body part that is being tested. Additionally, the user device 6 may store data and information from previous tests and compare a wearer's previous range of motion with his or her current range of motion.

Data and information from previous tests may also be stored remotely in a central database 22, for example and without limitation, in the cloud, and accessed by the user device 6 as necessary wirelessly. Data stored in a central database 22 may be available to the user device 6, any other user device 6 running a program or application capable of accessing the central database and processing the data, or other programs and users.

The user device 6 may also include one or more processors and/or protocols that direct, measure, display, and record real-time body part movement and biometric data. For example, the user device 6 protocols may direct a user to perform a series of movements with a body part placed within the monitoring sleeve 4, place the sleeve 4 on another corresponding body part, and perform a series of movements with that body part. The user device 6 may also direct a user to perform a series of movements on two or more body parts as described below and/or when more than one monitoring sleeve 4 is used. As the user moves the body part in the specified directions, the user device 6 may receive data from the sensor system 102 of the sleeve 4 that the user device 6 uses to determine the range of motion of the body part and record the information. The user device 6 may also produce a real-time display of the body part and monitoring sleeve 4 as the wearer moves his or her body part. Finally, the user device 6 protocols may record the data received from the sleeve 4 either as part of the application or remotely. The protocols may capture the complex movements of the body part within the sleeve 4 as a combination of angular rotation and direction. The protocols may use a set of landmarks, either internal or external to the user device 6, to control positioning error. The user device 6 may automatically detect and connect to any monitoring sleeves 4 in the vicinity of the user device 6.

The protocols may organize the reception and collection of data in any order or format. For example, in one embodiment the protocols may instruct a user to perform one particular range of motion movement at a time, record the data from that movement, and then instruct the user to perform another range of motion movement. In this embodiment, the range of motion movement may be the same or different than the previous movement. In another embodiment, the protocols may allow for a user to perform any movements he or she desires and record the data from those movements. The protocols may then instruct the user to perform particular movements if necessary. In yet another embodiment, the protocols may direct a user to perform a set of pre-specified movements and then record the data from those movements. In this embodiment, the practitioner or user may choose a set of protocols for the user to perform, or may allow the application to choose the protocols to run. Additionally, a practitioner may customize the protocols to suit the particular application, data, or analysis of range of motion needed. In any of the previously described embodiments, data may be recorded in a user device 6, cloud 20, and/or a central database 22. More than one user device 6 may also be used as a part of the system 2. Each of the user devices 6 of a system 2 may use the same or different protocols than the other user devices 6 of the same system 2.

In one embodiment, the wearer may be given one or more monitoring sleeves 4 on one or more body parts to be evaluate the range of motion of both injured and uninjured body parts by the practitioner. The practitioner may ensure proper anatomical placement of the monitoring sleeve 4. The practitioner may place the battery component 14 into the battery compartment 12 located on the lateral side of the sleeve 4. The battery life indicator 16 may be a light. The light may turn green when the battery 14 is the housing. The practitioner may then open the application on a user device 6 (iPad, tablet, smartphone) and/or project the application onto a display device 8 in the exam room. The practitioner may then select the range of motion assessment protocol to be used from a menu of options as described more fully below. The application may record the range of motion (in degrees), joint velocity (in degrees per second), and the force generated (in Newtons) in real-time and/or near real-time. The application may record a three dimensional rendering of the motion of the wearer's body parts and body that may be played back immediately or any time after each assessment. Range of motion data may be instantly recorded remotely, including but not limited to in a data cloud 20 or central database 22. Computations of the range of motion data may occur in the cloud 20 or as accessed in the central database 22 in order to take advantage of potentially greater computing power than available on the user device 6. The range of motion data may also be printed immediately after the time of the assessment. The wearer may visualize his or her progress with respect to range of motion through a personal and secure login to the application. Range of motion data may be displayed in a table within the application and may show the difference in range of motion. The wearer and/or practitioner may then, if necessary, select another range of motion assessment and follow the same sequence as described above.

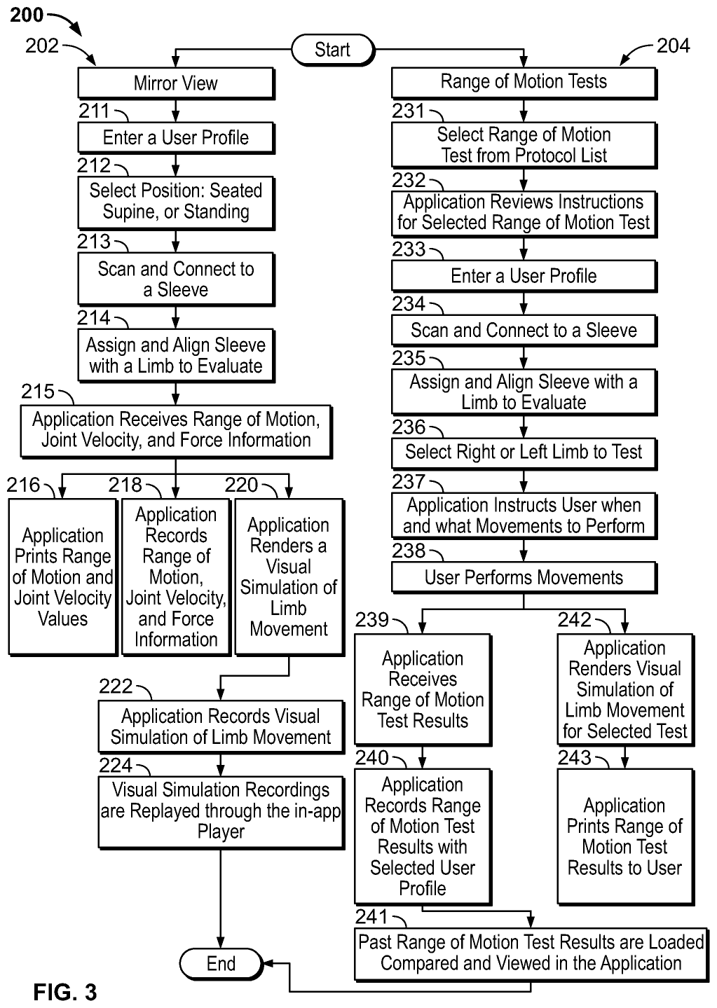

Referring now to FIG. 3, a method 200 of utilizing the system 2 is illustrated schematically according to example embodiments. As shown in FIG. 3, a practitioner may select between different operations, for example, using the user device 6. As shown in the illustrated embodiment, a practitioner may select between a mirror view 202 or a range of motion test 204 protocols on the user device 6.

The mirror view 202 may be used to collect and record free-form range of movement data, while the range of motion tests 204 instruct the wearer to perform certain movements. The software application may also take the wearer through a video tutorial which may be bypassed if the wearer is familiar with the range of motion assessment.

If the practitioner selects the mirror view 202, the protocol may follow the following steps. At 211, the practitioner may be asked to enter a user profile for the wearer into the user device 6. Next, at 212, the practitioner may be asked to select a position for the movements using the user device 6. At 212, the choices of positions may include, but is not limited to, seated, supine, or standing. Next, at 213, the user device 6 may scan and connect to at least one, but potentially more than one, monitoring sleeve 4. Then, at 214, the practitioner may assign and align monitoring sleeves 4 with the respective body parts to be evaluated. The wearer may then, at 215, move the body parts to be evaluated, and the application in the user device 6 may receive data, including but not limited to range of motion, velocity, and force data from the monitoring sleeves 4. The application may then (at 216) print the data received, (at 218) record the data received on the user device 6/cloud 20/or central database 22, and (at 220) render a visual representation of the movement. Next, at 222, the application may record the visual representation of movement and (at 224) replay the visual representation on the user device 6 and/or the display device 8.

Alternatively, if the practitioner selects range of motion tests, the protocol may operate as follows. First, at 231, the practitioner may select a specified range of motion test desired from a list of available tests. Next, at 232, the application may provide the instructions for the range of motion test selected at 231, for example, via the display device 8. The practitioner may then, at 233, be asked to enter a user profile for the wearer using the user device 6. The practitioner may then, at 234, scan and connect the user device 6 to a monitoring sleeve 4 and, at 235, assign and align the monitoring sleeve 4 with the body parts to be evaluated. Next, at 236, the practitioner may utilize the user device 6 to select either the body part or parts to test as necessary (e.g., selection between right or left limb for testing). The application then, at 237, instructs the wearer on what motions to perform and when to perform them by displaying the instructions on the display device 8. At 238, the wearer of the sleeve 4 performs those movements per the instructions provided at 237. The user device 6 may, at 239, receive the range of motion test results. Also, at 240, the test results may be recorded in memory on the user device 6, cloud 20, and/or central database 22. Furthermore, at 241, processors at the user device 6 may access previously-stored range of motion test results and compare those with the new test results (received at 239) in the application on the user device 6. The application, at 242, may also render a visual representation of the body part movement for the selected test and display the visual representation either on the user device 6 or display device 8. The application may also, at 243, print the range of motion test results received at 239.

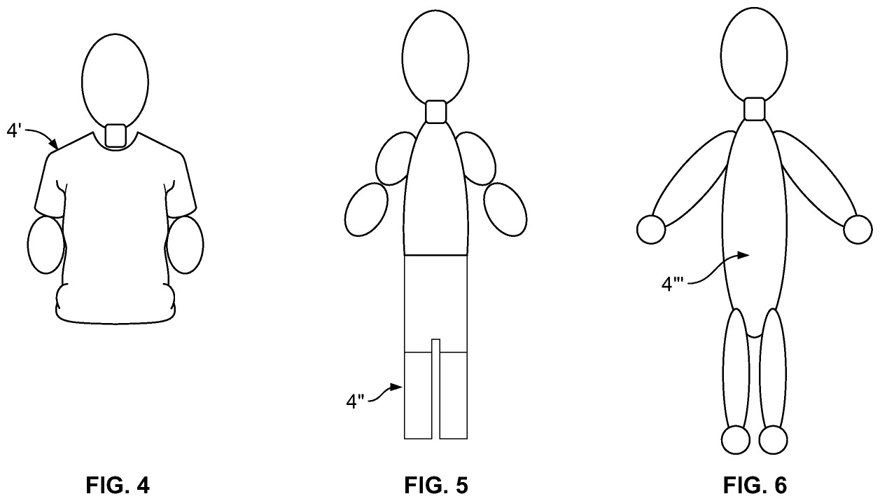

In another embodiment of an Objective Range of Motion Monitoring System shown in FIG. 4, the monitoring sleeve 4′ may be formed as a shirt substantially covering a torso of a human being. The monitoring sleeve 4′ formed as a shirt may be configured to measure the range of motion of a human spine in addition to the arms and/or shoulders of a wearer. In another embodiment shown in FIG. 5, the monitoring sleeve 4″ may be formed as a part of pants or leggings configured to measure the range of motion of a lower spine and/or pelvis in addition to the legs and/or hips of the wearer. In yet another embodiment shown in FIG. 6, the monitoring sleeve 4′″ may be formed as a body suit which substantially covers a human body. The body suit may be configured to measure the range of motion of any portion of the human body and the range of motion of a human body overall.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the present disclosure. It is understood that various changes may be made in the function and arrangement of elements described in an exemplary embodiment without departing from the scope of the present disclosure as set forth in the appended claims.

1. A motion monitoring system comprising:
a wearable monitoring device configured to be worn on a human body that includes a body part, the wearable monitoring device being deformable with movement of the body part, the wearable monitoring device including a sensor system configured to deform with movement of the body part, the sensor system configured to detect deformation occurring with movement of the body part; and
a user device that is in communication with the wearable monitoring device, the user device being capable of receiving data from the sensor system that corresponds to the detected deformation occurring with movement of the body part.

2. The motion monitoring system of claim 1, further comprising a display device configured to visually render movement of the body part according to the data received from the sensor system corresponding to the detected deformation occurring with movement of the body part.

3. The motion monitoring system of claim 2, wherein the display device is wirelessly connected to the wearable monitoring device.

4. The motion monitoring system of claim 2, wherein the sensor system includes a lattice that includes an array of repeating geometric patterns, the lattice being flexible and deformable and electrically conductive, the lattice including at least one first sensor configured to detect deformation of at least one of the geometric patterns occurring with movement of the body part; and
wherein the display device is configured to visually render movement of the body part according to the detected deformation of the at least one of the geometric patterns.

5. The motion monitoring system of claim 4, wherein the at least one first sensor is configured to detect deformation of the at least one of the geometric patterns by measuring a change in voltage across the at least one first sensor; and
wherein the display device is configured to visually render movement of the body part according to the measured change in voltage.

6. The motion monitoring system of claim 4, wherein the at least one lattice includes at least one second sensor configured to detect a second characteristic that is different from the deformation detected by the at least one first sensor.

7. The motion monitoring system of claim 6, wherein the first sensor is configured to power and transmit data for the at least one second sensor.

8. The motion monitoring system of claim 1, wherein the wearable monitoring device is a sleeve that wraps about the body part and that is deformable with movement of the body part.

9. The motion monitoring system of claim 1, wherein the sensor system is configured to contact the skin of the body part.

10. The motion monitoring system of claim 1, wherein the user device is in wireless communication with the wearable monitoring device for wirelessly receiving the data from the sensor system.

11. A method for determining the range of motion of a human body part, the method comprising:
detecting, by a sensor system of a monitoring device that is worn on a human body that includes a body part, deformation of the sensor system occurring with movement of the body part;
receiving data by a user device from a wearable monitoring device, the data corresponding to the detected deformation occurring with movement of the body part; and
visually rendering, with a display device, movement of the body part according to the received data.

12. The method of claim 11, further comprising detecting, by the sensor system, an angular rotation and a direction of the monitoring device;
wherein receiving the data includes receiving the data that corresponds to the detected angular rotation and the direction of the monitoring device.

13. The method of claim 11, further comprising calculating a range of motion of the body part according to the received data.

14. The method of claim 13, further comprising saving the range of motion in computerized memory.

15. The method of claim 14, further comprising:
determining a first range of motion of the body part in a first range of motion evaluation of the body part;
determining a second range of motion of the body part in a second range of motion evaluation of the body part; and
comparing the determined first range of motion to the determined second range of motion.

16. The method of claim 11, further comprising directing a user to move the body part in a particular movement.

17. The method of claim 11, wherein the sensor system includes a lattice that includes an array of repeating geometric patterns, the lattice being flexible and deformable and electrically conductive;
wherein detecting deformation of the sensor system includes detecting, by at least one first sensor of the lattice, deformation of at least one of the geometric patterns occurring with movement of the body part; and
wherein visually rendering movement of the body part includes visually rendering movement of the body part according to the detected deformation of the at least one of the geometric patterns.

18. The method of claim 17, wherein detecting deformation includes detecting, by the at least one first sensor, deformation of the at least one of the geometric patterns by measuring a change in voltage across the at least one first sensor; and
wherein visually rendering movement includes visually rendering movement of the body part according to the measured change in voltage.

19. The method of claim 17, wherein the at least one lattice includes at least one second sensor; and
further comprising detecting, by the at least one second sensor, a second characteristic that is different from the deformation detected by the at least one first sensor.

20. The method of claim 19, further comprising the at least one first sensor powering and transmitting data for the at least one second sensor.
